(19) United States
(12) Patent Application Publication
(10) Pub. No.: US 2025/0277164 A1
(43) Pub. Date: Sep. 4, 2025

(54) PLASTIFICANTE NATURAL DERIVADO DE ESTERES GRAXOS COM ALTO CONTEUDO DE EPOXI

(71) Applicant: BRASIL BIO FUELS S.A, SÃO JOÃO DA BALIZA (BR)

(72) Inventor: Setsuo Sato, Piracicaba (BR)

(21) Appl. No.: 18/596,105

(22) Filed: Mar. 5, 2024

(30) Foreign Application Priority Data
Mar. 1, 2024 (BR) 10 2024 004250 6

(51) Int. Cl. C11C 1/00; C11C 1/02

(52) U.S. Cl. CPC C11C 1/002; C11C 1/02

(57) ABSTRACT

Some phthalates are considered toxic and carcinogenic, which limits their use and demand for safer and more environmentally friendly plasticizers. The development of plasticizers produced from vegetable oil derivatives with a high degree of unsaturation is described, which allows the production of esters with a high epoxy content, guaranteeing excellent performance in terms of plasticization, high compatibility with polymeric materials such as polyvinyl chloride, polyesters, nitrocellulose, thereby avoiding migration when used in polymeric matrices. Fatty materials produced from linseed or tung oil have a high degree of unsaturation compared to vegetable oils such as soybean, corn, peanut, canola, sunflower; however, they are scarce and expensive when compared to them. we start Starting from common oils such as soybean and similar oils and separate the saturated part, generates materials with a high degree of unsaturation similar to fatty materials derived from linseed oil.

Plasticized polyvinyl chloride polymers have been extensively used throughout the world in construction applications, piping, cables, flooring, footwear, bags, synthetic leather, food protection films, and various packaging. Phthalates are the most used plasticizers, but some phthalates are considered toxic and carcinogenic, which limits their use and increases the demand for safer and more environmentally friendly plasticizers.

The invention is based on the development of plasticizers produced from vegetable oil derivatives with a high degree of unsaturation, which allows the production of esters with a high epoxy content, guaranteeing excellent performance in terms of plasticization, high compatibility with polymeric materials such as polyvinyl chloride, polyesters, nitrocellulose thus avoiding migration when used in these polymeric matrices.

Fatty materials produced from linseed or tung oil have a high degree of unsaturation compared to vegetable oils such as soybean, corn, peanut, canola, sunflower; however, they are scarce and expensive when compared to them. In this invention we start from common oils such as soybean and similar oils and separate the saturated part, generating materials with a high degree of unsaturation similar to fatty materials derived from linseed oil.

### Status Quo

Dioctylphthalate, dibutylphthalate, diisononylphthalate and other phthalic derivatives are being replaced by other products considered non-phthalic such as dicyclohexylphthalate (DCHP) which, although it is still a phthalate, does not contain the aromatic ring and is therefore considered safe. It is a more efficient plasticizer in replacing phthalates, but it is expensive because its processing is extremely expensive. Other products such as terephthalate derivatives are being used, but all of them are of petrochemical origin.

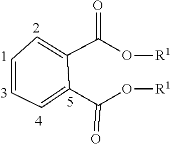

### Chemical Structure of Phthalates

There is a diversity of plasticizers considered vegetable, such as epoxidized esters of vegetable oils, cardanol derivatives, citrates, rosinic acid esters (their synthesis is extremely complex), glycidol esters. All of them have their advantages and disadvantages, but in most cases they are used with other plasticizers to compensate for performance. The figures below show the chemical structure of these products:

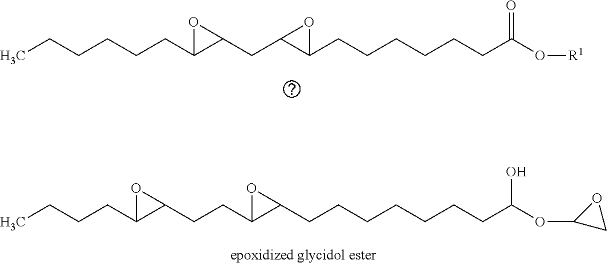

Patent WO2013003225A2 deals exactly with the products of the above structures, more specifically the epoxidized esters derived from soybean and corn oil.

The article “Designing of Green Plasticizers and Assessment of the Effectiveness of Their Use” deals with the use of adipic acid esters as a substitute for phthalates specifically in materials for hospital use such as syringes, blood transfusion tubes and of intravenously application of medicament.

There is a growing effort in industry and academia to find and develop safer, more efficient and more economically viable plasticizers.

The invention below is another option we have proposed for this market segment.

### DESCRIPTION OF THE INVENTION

The first part of the development was the separation of saturated chains via crystallization and filtration.

| CARBON | STANDARD | APÓS CRISTALIZAÇÃO |
|---|---|---|
| CHAIN | % | % |
| C16 | 11.3 | 3.1 |
| C18:0 | 4.7 | 2.0 |
| C18:1 | 23.2 | 26.2 |
| C18:2 | 54.7 | 61.8 |
| C18:3 | 6.1 | 6.9 |

An important point to comment is that if we simply look at the content of saturated chains, there is tallow fatty acid (TOFA) which has a similar saturated content, but the concentration of linoleic acid (2 unsaturations) is 39% versus 61.8% of this inventive process, which ensures greater formation of oxirane rings.

The second stage was the production of the respective esters whose strategy was to have products with high polarity to be extremely compatible with the polymer chains and also with sufficient molecular weight so that they do not migrate.

The third step was the synthesis of epoxidized products via the classic route using hydrogen peroxide and peracids as catalyst.

Below are the products developed according to the mentioned technique:

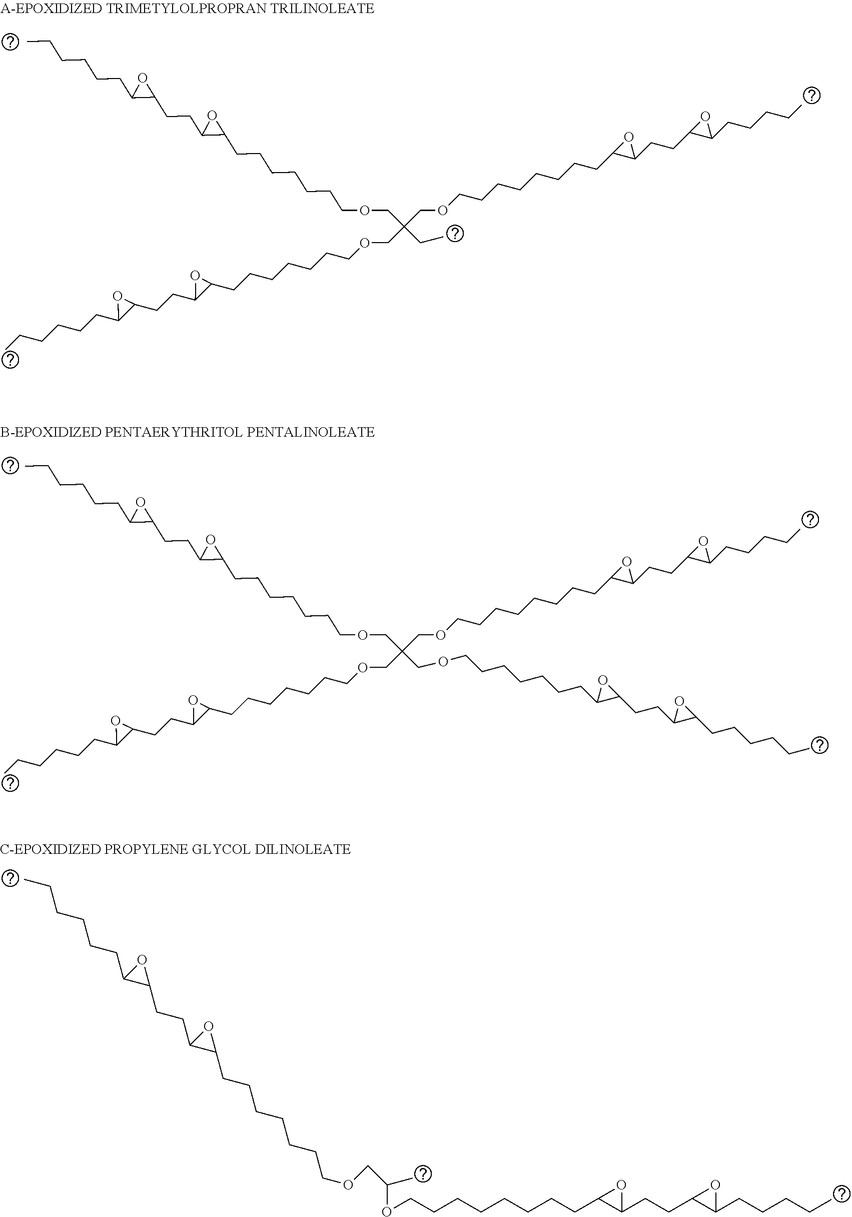

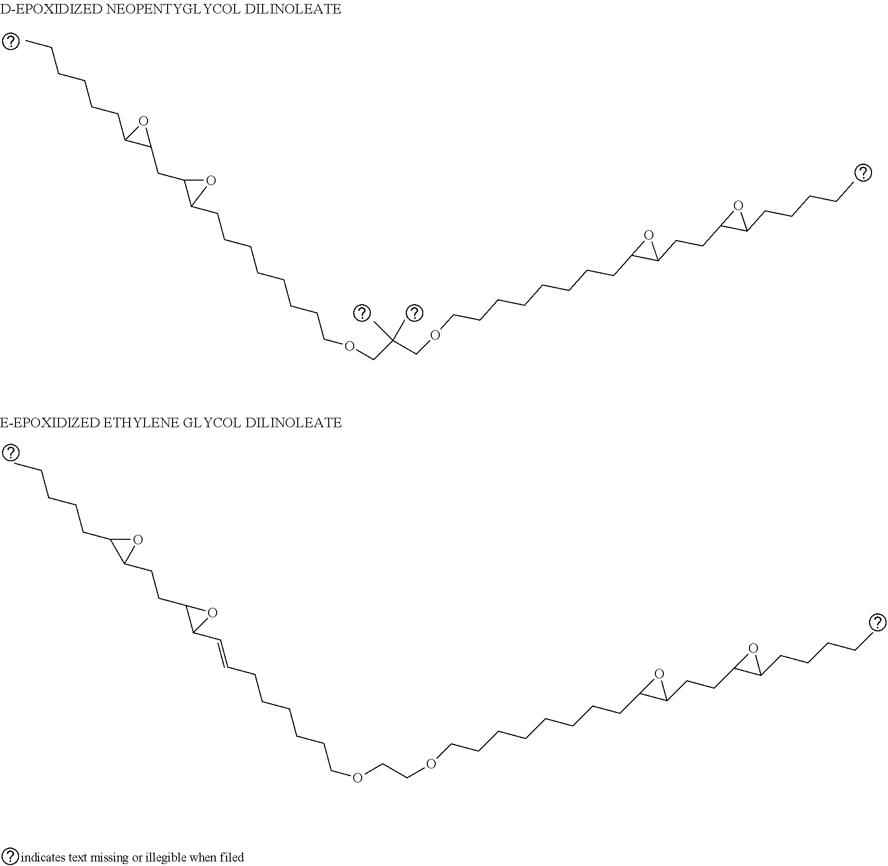

In this sense, the following are described:
1—Preparation of fatty acids with a high unsaturation content, with a limited content of saturated fatty acids having a content between 1 and 15%, preferably in the range of 2 to 8%.
2—Product having chemical structure A, where the product is epoxidized trimethylolpropane trilinoleate.
3—Product having the chemical structure C, where the product is epoxidized propylene glycol dilinoleate, or its similar dipropylene glycol, tripropylene glycol, tetrapropylene glycol and pentapropylene glycol.
4—Product having chemical structure D, where the product is epoxidized neopentylglycol dilinoleate.
5—Product having the chemical structure E, where the product is epoxidized ethylene glycol dilinoleate, or its similar diethylene glycol, triethylene glycol, tetraethylene glycol and pentaethylene glycol.
6—Use of products as defined in items 2 to 5 for the production of flexible polyvinyl chloride (PVC) films, footwear compounds, flexible PVC for application producing floors, upholstery, bag items (synthetic leather).
7—Use of products as defined in items 2 to 5 in paints and varnishes.
8—Use of products as defined in items 2 to 5 in synthetic nail enamel.

1. Preparation of fatty acids with high unsaturation content, with limited content of saturated fatty acids characterized by the fact that it has a content between 1 and 15%, preferably in the range of 2 to 8%.

2. Product characterized by the fact that it has the chemical structure A: 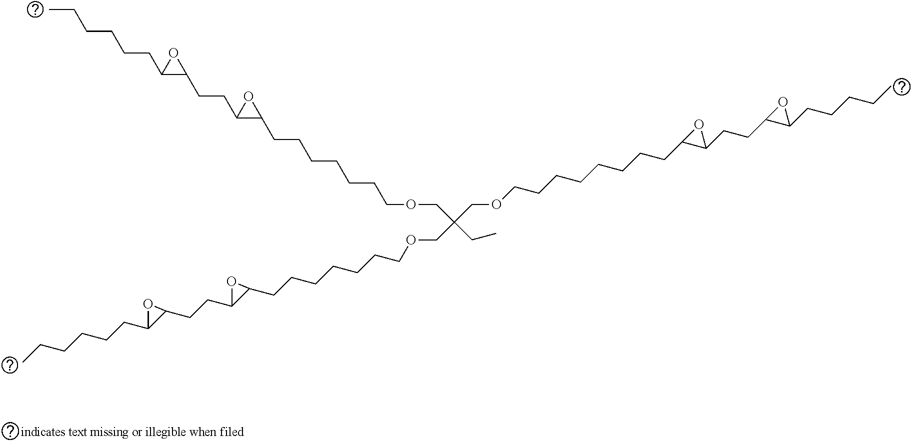 [SMILES: CCCCCCC1OC1CCC1OC1CCCCCCCOCC(CC)(COCCCCCCCCC1OC1CCC1OC1CCCCC)COCCCCCCCC1OC1CCC1OC1CCCCCC]
wherein the product is epoxidized trimethylolpropane trilinoleate; or the chemical structure C: 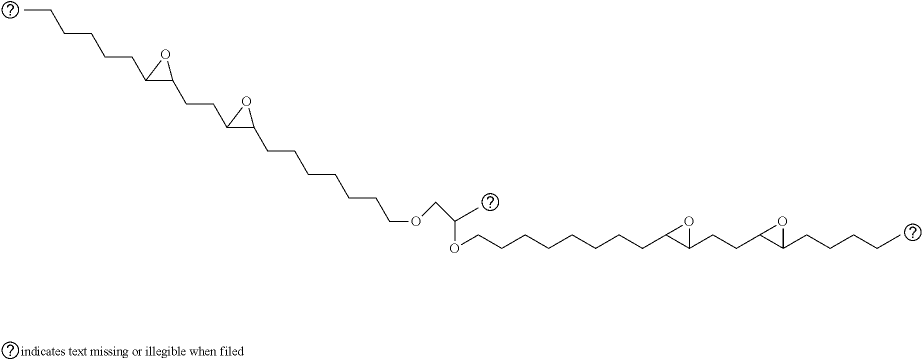 [SMILES: CCCCCCC1OC1CCC1OC1CCCCCCCOCC(C)OCCCCCCCCC1OC1CCC1OC1CCCCC]
wherein the product is epoxidized proprylene glycol dilinoleate, or its similar dipropylene glycol, tripropylene glycol, tetrapropylene glycol and pentapropylene glycol; or the chemical structure D: 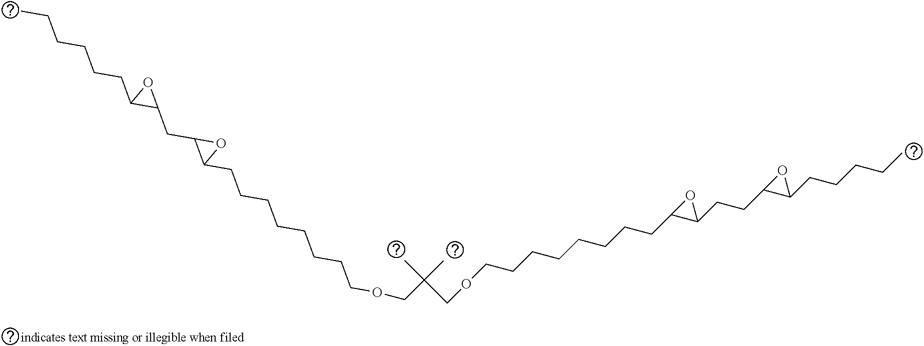 [SMILES: CCCCCCC1OC1CC1OC1CCCCCCCCOCC(C)(C)COCCCCCCCCC1OC1CCC1OC1CCCCC]
wherein the product is epoxidized neopentylglycol dilinoleate; or the chemical structure E: 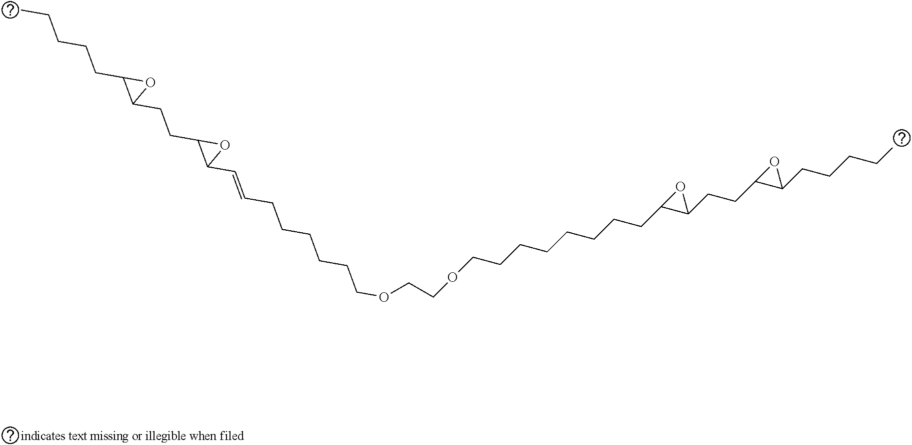 [SMILES: CCCCCC1OC1CCC1OC1/C=C/CCCCCCOCCOCCCCCCCCC1OC1CCC1OC1CCCCC]
wherein the product is epoxidized ethylene glycol dilinoleate, or its similar diethylene glycol, triethylene glycol, tetraethylene glycol and pentaethylene glycol.

3-5. (canceled)

6. Use of the products as defined claim 2, characterized by being for the production of flexible polyvinyl chloride (PVC) films, footwear compounds, flexible PVC for application producing floors, upholstery, bag articles (synthetic leather).

7. Use of the products as defined in claim 2, characterized by being in paints and varnishes.

8. Use of the products as defined in claim 2, characterized by being in synthetic nail enamel.
